(19) United States
(12) Patent Application Publication
(10) Pub. No.: US 2015/0320362 A1
(43) Pub. Date: Nov. 12, 2015

(54) EXERCISE EFFECT ANALYSIS SYSTEM

(71) Applicant: HITACHI, LTD., Chiyoda-ku, Tokyo (JP)

(72) Inventors: Yasutaka HASEGAWA, Tokyo (JP); Takanobu OSAKI, Tokyo (JP); Kazuyuki SHIMADA, Tokyo (JP); Hideyuki BAN, Tokyo (JP)

(21) Appl. No.: 14/649,748

(22) PCT Filed: Dec. 20, 2012
(86) PCT No.: PCT/JP2012/083084

§ 371 Date: Jun. 4, 2015

(51) Int. Cl. A61B 5/00; A61B 5/08

(52) U.S. Cl. CPC A61B 5/7275; A61B 5/7475; A61B 5/742; A61B 5/08

(57) ABSTRACT

The invention is an exercise effect analysis system for analyzing and displaying an exercise effect on the basis of lung function information of a user. The exercise effect analysis system includes: an input unit for inputting at least exercise information and lung age information of the user; an exercise amount change calculation unit for calculating an exercise amount change amount that is a difference between first exercise information and second exercise information inputted in the input unit; a lung age change calculation unit for calculating a lung age change amount that is a difference between first lung age information and second lung age information; and an output unit for displaying the lung age change amount and at least one of the exercise information of the user and the exercise amount change amount.

### TECHNICAL FIELD

The present invention relates to an exercise effect analysis system for analyzing and displaying an effect of exercise on the basis of lung function information of a user.

### BACKGROUND ART

In order to perform and continue exercise, it is important to show an effect of exercise. Indexes for indicating an exercise effect are a pulse, maximal oxygen uptake, and the like. PTL 1 discloses an apparatus for displaying a physical strength determination value (physical strength age) on the basis of a pulse, maximal oxygen uptake, and age.

### CITATION LIST

### Patent Literature

PTL 1: JP-A-2010-233677

### SUMMARY OF INVENTION

### Technical Problems

However, in order to measure a pulse and maximal oxygen uptake, an exercise load is needed. Therefore, the pulse and the maximal oxygen uptake cannot be easily measured. Meanwhile, there is a lung age (calculated based on forced expiratory volume in 1 second, sex, and age), which is not intended to measure an exercise effect, as a kind of lung function information for use in early detection/prevention of COPD (chronic obstructive pulmonary disease). The lung age is measured by an apparatus called spirometry and can be measured from a respiration. However, conventionally, a system for effectively analyzing and displaying an exercise effect on the basis of lung age information has not been considered.

The invention provides an exercise effect analysis system for effectively analyzing and displaying an effect of exercise on the basis of lung age information of a user and assisting the user to perform and continue exercise.

### Solution to Problems

In order to solve the above problems, for example, configurations described in Claims are employed. The present application includes a plurality of means to solve the above problems, and, as an example thereof, an exercise effect analysis system for analyzing and displaying an exercise effect on the basis of lung function information of a user is provided. The exercise effect analysis system includes: an input unit for inputting at least exercise information and lung age information of the user; an exercise amount change calculation unit for calculating an exercise amount change amount that is a difference between first exercise information and second exercise information inputted in the input unit; a lung age change calculation unit for calculating a lung age change amount that is a difference between first lung age information and second lung age information; and an output unit for displaying the lung age change amount and at least one of the exercise information of the user and the exercise amount change amount.

As another example, an exercise effect analysis system includes: an input unit for inputting at least exercise information and lung age information of a user; an exercise amount change calculation unit for calculating an exercise amount change amount that is a difference between first exercise information and second exercise information inputted in the input unit; a lung age change calculation unit for calculating a lung age change amount that is a difference between first lung age information and second lung age information; a subtraction calculation unit for calculating a difference between a lung age and an actual age, the difference being a difference between age information and the lung age information of the user; a storage device in which user recording information in which at least the exercise information and the lung age information of the user are recorded, an estimation formula showing a relationship among the exercise information, the age, the lung age, and an estimated lung age change amount, and lung age error distribution information indicating distribution of errors of lung age change amounts of a plurality of users are stored; a lung age error range calculation unit for substituting the difference between the lung age and the actual age for the estimation formula to calculate an estimated lung age and calculating a lung age error range on the basis of the estimated lung age and the lung age error distribution information; a lung age improvement determination unit for comparing the lung age change amount with the lung age error range to determine improvement of the lung age of the user; and an output unit for displaying the lung age change amount, the lung age error range, and at least one of the exercise information of the user recording information and the exercise amount change amount.

As still another example, an exercise effect analysis system includes: an input unit for inputting at least exercise information and lung age information of a user; an exercise amount change calculation unit for calculating an exercise amount change amount that is a difference between first exercise information and second exercise information inputted in the input unit; a lung age change calculation unit for calculating a lung age change amount that is a difference between first lung age information and second lung age information; a weight change calculation unit for calculating a weight change amount that is a difference between first weight information and second weight information; a subtraction calculation unit for calculating a difference between a lung age and an actual age, the difference being a difference between age information and the lung age information of the user; a storage device in which user recording information in which at least the exercise information, the weight information, and the lung age information of the user are recorded, an estimation formula showing a relationship among the exercise information, the weight information, the age, the lung age, and an estimated lung age change amount, and lung age error distribution information indicating distribution of errors of lung age change amounts of a plurality of users are stored; a lung age error range calculation unit for substituting the difference between the lung age and the actual age for the estimation formula to calculate an estimated lung age and calculating a lung age error range on the basis of the estimated lung age and the lung age error distribution information; a lung age improvement determination unit for comparing the lung age change amount with the lung age error range to determine improvement of the lung age of the user; and an output unit for displaying the lung age change amount, the lung age error range, and at least one of the exercise information of the user recording information and the exercise amount change amount.

### Advantageous Effects of Invention

According to the invention, by inputting exercise information and lung age information that can be easily measured by a user, it is possible to accurately determine improvement of a lung age caused by exercise. This makes it possible to assist the user to perform and continue exercise.

Further features related to the invention will become apparent from the description of this specification and accompanying drawings. Further, problems, configurations, and effects other than the above-mentioned ones will become apparent from the following description of an example.

### BRIEF DESCRIPTION OF DRAWINGS

FIG. 1 shows an example of a functional block configuration of an exercise effect analysis system.

FIG. 2 shows an example of a relationship among a lung age, an exercise time period, and weight.

FIG. 3 is a flowchart showing a flow in which information is inputted when the exercise effect analysis system is started.

FIG. 4 is a flowchart showing a flow from input of user information to determination of improvement of a lung age.

FIG. 5 is a flowchart showing a flow from input of medical examination data to calculation of lung age error distribution.

FIG. 6 shows an example of information managed in a medical examination data management unit.

FIG. 7 shows an example of information managed in a recording data management unit.

FIG. 8 shows examples of estimation formulae managed in an estimation formula management unit.

FIG. 9 shows an example of lung age error distribution data managed in a lung age error distribution management unit.

FIG. 10 shows an example of a user information input screen displayed in an output unit at the time of starting.

FIG. 11 shows an example of a recording screen displayed in the output unit at the time of recording.

### DESCRIPTION OF EMBODIMENT

Hereinafter, an example of the invention will be described with reference to accompanying drawings. Note that the accompanying drawings show a specific example in accordance with principles of the invention. Those drawings are used to understand the invention but are never used to limitedly interpret the invention.

### <Configuration of System>

An exercise effect analysis system according to the example includes an information processing apparatus such as a workstation or a personal computer. The information processing apparatus includes a central processor, a storage unit such as a memory, and a storage medium. The central processor is configured by a processor such as a CPU (Central Processing Unit). The storage medium is, for example, a non-volatile storage medium. Examples of the non-volatile storage medium include a magnetic disk and a non-volatile memory. In the storage medium, a program for realizing a function of the exercise effect analysis system, a calculation result obtained when the program is executed, and the like are stored. In the memory, the program stored in the storage medium is loaded. The CPU executes the program loaded in the memory. Therefore, each processing unit of the exercise effect analysis system described below is realized as a program to be executed in a computer. Note that a configuration of the example may be realized with the use of hardware by, for example, designing a part or all thereof with an integrated circuit.

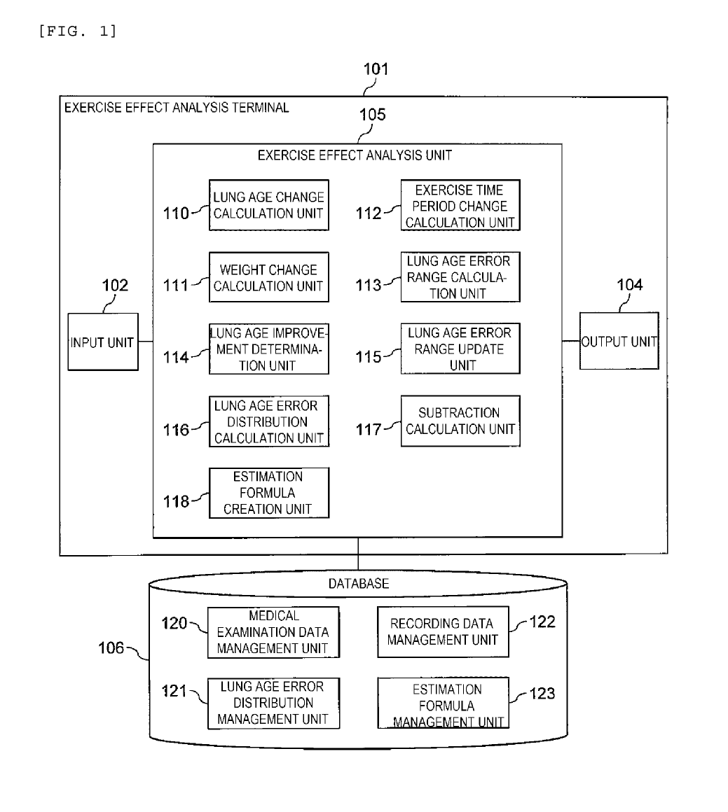

FIG. 1 shows a configuration diagram of an exercise effect analysis system according to this example. The exercise effect analysis system includes an exercise effect analysis terminal 101 and a database 106. The exercise effect analysis terminal 101 includes an input unit 102, an exercise effect analysis unit 105, and an output unit 104. As the input unit 102, a mouse, a keyboard, or the like is used. As the output unit 104, a display for displaying information inputted with the use of the input unit 102 and a calculation result of the exercise effect analysis unit 105, a printer for printing the inputted information and the calculation result, or the like is used. In this example, the exercise effect analysis unit 105 is realized as a program to be executed in the computer as described above. The program and the database 106 are stored in the storage medium.

The exercise effect analysis unit 105 includes a lung age change calculation unit 110, a weight change calculation unit 111, an exercise time period change calculation unit 112, a lung age error range calculation unit 113, a lung age improvement determination unit 114, a lung age error range update unit 115, a lung age error distribution calculation unit 116, a subtraction calculation unit 117, and an estimation formula creation unit 118.

The database 106 includes a medical examination data management unit 120, a lung age error distribution management unit 121, a recording data management unit 122, and an estimation formula management unit 123. Note that, in the following description, information of each of the management units 120, 121, 122, and 123 in the database 106 will be described with the use of a “table” structure. However, the information is not necessarily expressed by a table data structure, and can be expressed by a list, a cue, or another format data structure or another form. Therefore, in order to show that the information does not depend on the data structure, “table”, “list”, “DB”, “cue”, and the like are merely referred to as “information” in some cases.

The lung age change calculation unit 110 calculates a lung age reduction amount on the basis of a lung age of a user inputted in the input unit 102. The weight change calculation unit 111 calculates a weight reduction amount on the basis of weight of a user inputted in the input unit 102. The exercise time period change calculation unit 112 calculates an exercise time period increase amount on the basis of an exercise time period of a user inputted in the input unit 102. The subtraction calculation unit 117 calculates a difference between a lung age and an actual age of a user on the basis of the lung age and and the actual age inputted in the input unit 102.

The lung age error range calculation unit 113 calculates a lung age error range on the basis of the calculated difference between the lung age and the actual age, a lung age reduction amount estimation formula 801 and a lung age estimation formula 803 (see FIG. 8) managed in the estimation formula management unit 123, and lung age error distribution managed in the lung age error distribution management unit 121.

The lung age improvement determination unit 114 determines whether or not the lung age is improved on the basis of the calculated lung age error range and the lung age reduction amount calculated in the lung age change calculation unit 110. In a case where the lung age improvement determination unit 114 determines that the lung age is improved, the lung age error range update unit 115 updates the lung age error range calculated in the lung age error range calculation unit 113.

The estimation formula creation unit 118 analyzes a relationship among a lung age reduction amount, a weight reduction amount, an exercise time period increase amount, and a difference between a lung age and an actual age, which are calculated on the basis of medical examination information of a plurality of persons for a plurality of number of times managed in the medical examination data management unit 120 and creates a lung age reduction amount estimation formula, a weight reduction amount estimation formula, and a lung age estimation formula. Details of those estimation formulae will be described below.

The lung age error distribution calculation unit 116 analyzes the relationship among the lung age reduction amount, the weight reduction amount, the exercise time period increase amount, and the difference between the lung age and the actual age on the basis of the medical examination information managed in the medical examination data management unit 120 and calculates the lung age error distribution. Details of this lung age error distribution information will be described below.

### <Relationship Among Lung Age, Exercise Time Period, and Weight>

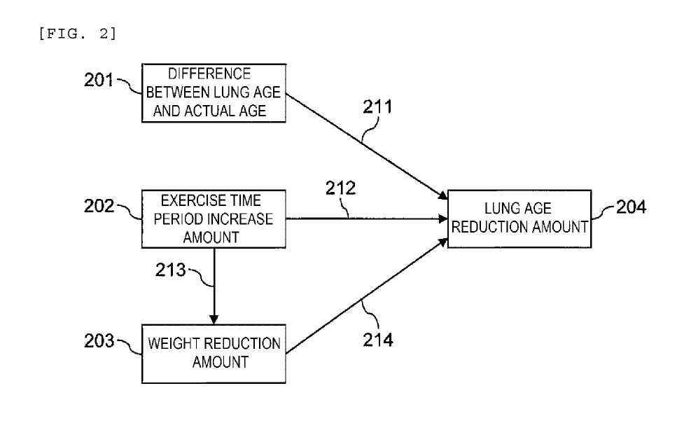

FIG. 2 shows an example of a relationship among a lung age, an exercise time period, and weight. FIG. 2 shows an influence relationship between a difference 201 between a lung age and an actual age, an exercise time period increase amount 202, and a weight reduction amount 203 and a lung age reduction amount 204.

An influence 211 of the difference 201 between the lung age and the actual age on the lung age reduction amount 204 indicates that the lung age reduction amount 204 is increased as the difference 201 between the lung age and the actual age is increased. This relationship indicates that the lung age is reduced due to the difference between the lung age and the actual age which has no relation to exercise, and therefore it is necessary to remove the relationship to determine improvement of the lung age caused by the exercise.

An influence 212 of the exercise time period increase amount 202 on the lung age reduction amount 204 indicates that the lung age reduction amount 204 is increased as the exercise time period increase amount 202 is increased. In other words, the influence 212 indicates that exercise directly influences the lung age reduction amount. This relationship can be explained as follows: respiratory muscle such as intercostal muscle is trained by exercise to improve a lung function and reduce the lung age.

An influence 213 of the exercise time period increase amount 202 on the weight reduction amount 203 and an influence 214 of the weight reduction amount 203 on the lung age reduction amount 204 indicate that, as the exercise time period increase amount 202 is increased, the weight reduction amount 203 is increased and the lung age reduction amount 204 is further increased. This relationship indicates that exercise indirectly influences the lung age reduction amount via the weight. This can be explained as follows: increase in exercise leads to reduction in weight (reduction in visceral fat) and therefore a diaphragm that is respiratory muscle is smoothly contracted, and, as a result, the lung function is improved to reduce the lung age. Those influence relationships can be found by acquiring and analyzing the lung age, the exercise time period, the weight, and the age from the medical examination information managed in the medical examination data management unit 120. Specific analysis processing thereof will be described below.

### <Configuration of Database>

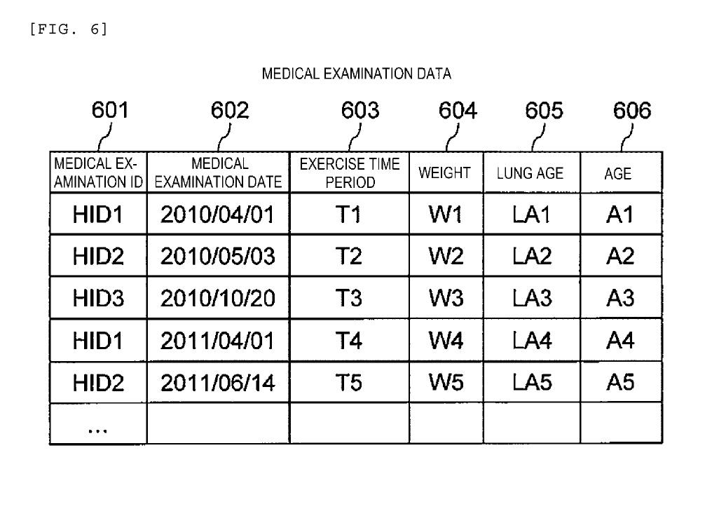

Information managed in the database 106 will be described with reference to drawings. FIG. 6 shows an example of information managed in the medical examination data management unit 120. Regarding the medical examination information of the plurality of persons for the plurality of number of times, the medical examination data management unit 120 manages the medical examination information such as the lung age, the weight, the exercise time period, and the age for each medical examination ID (or individually) and for each medical examination date. Specifically, the medical examination data management unit 120 includes, as components, a medical examination ID 601 for uniquely identifying medical examination, medical examination date 602, an exercise time period 603 asked at the medical examination date, weight 604 measured at the medical examination date, a lung age 605 measured at the medical examination date, and an age 606 at the medical examination date.

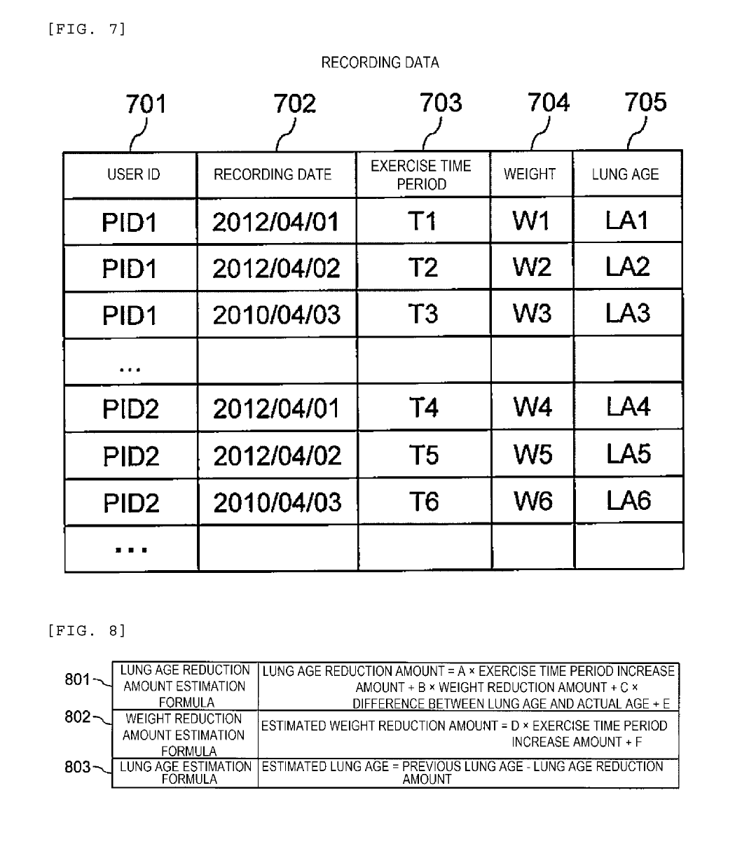

FIG. 7 shows an example of information managed in the recording data management unit 122. The recording data management unit 122 manages the daily exercise time period, lung age, and weight measured by a user. Specifically, the recording data management unit 122 includes, as components, a user ID 701 for uniquely identifying a user, recording date 702, an exercise time period 703, weight 704, and a lung age 705. The exercise time period 703, the weight 704, and the lung age 705 are managed for each user ID 701 and for each recording date 702.

FIG. 8 shows examples of estimation formulae managed in the estimation formula management unit 123. The estimation formula management unit 123 manages the lung age reduction amount estimation formula, the weight reduction amount estimation formula, and the lung age estimation formula created by the estimation formula creation unit 118. Those estimation formulae are numerical formulae indicating the relationship among the lung age, the exercise time period, and the weight in FIG. 2 described above.

In the estimation formula management unit 123, the following formula is stored as the lung age reduction amount estimation formula 801 that estimates the lung age reduction amount on the basis of the exercise time period increase amount, the weight reduction amount, and the difference between the lung age and the actual age (previous lung age−actual age).

Lung age reduction amount=A×exercise time period increase amount+B×weight reduction amount+C×difference between lung age and actual age+E

Further, in the estimation formula management unit 123, the following formula is stored as a weight reduction amount estimation formula 802 for estimating the weight reduction amount from the exercise time period increase amount.

Estimated weight reduction amount=D×exercise time period increase amount+F

Furthermore, in the estimation formula management unit 123, the following formula is stored as the lung age estimation formula 803 for estimating the lung age from the previous lung age and the lung age reduction amount.

Estimated lung age=previous lung age−lung age reduction amount

Note that, in FIGS. 8, A, B, C, and D indicate regression coefficients and E and F indicate constant terms.

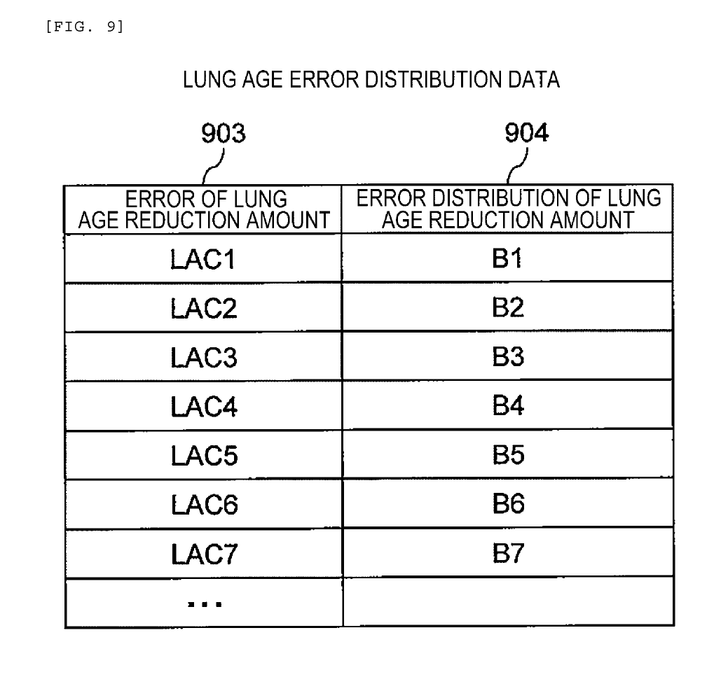

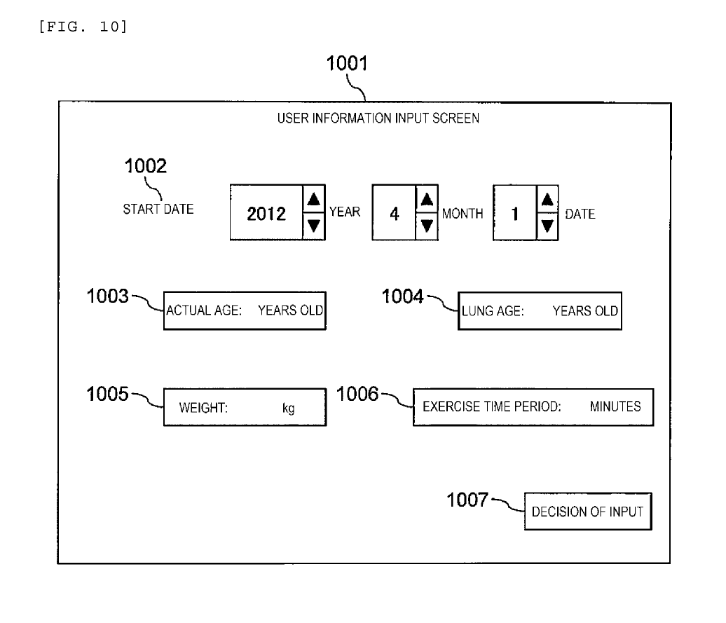

FIG. 9 shows an example of information on the lung age error distribution managed in the lung age error distribution management unit 121. The lung age error distribution management unit 121 manages the lung age error distribution calculated in the lung age error distribution calculation unit 116. Specifically, the lung age error distribution management unit 121 includes, as components, an error 903 of the lung age reduction amount and a distribution 904 of the error of the lung age reduction amount. The error 903 of the lung age reduction amount is a value of the difference between the lung age and the actual age which has no relation to exercise. Further, the distribution 904 of the error of the lung age reduction amount indicates a value (%) obtained by dividing a degree of the error 903 of the lung age reduction amount for each value by a total degree.

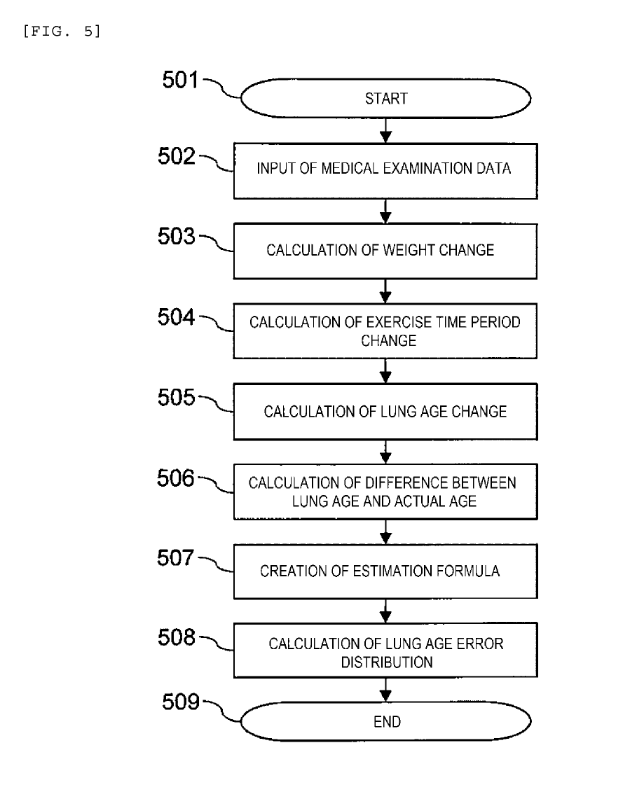

For example, in a case where the values of the error 903 of the lung age reduction amount are LAC1=−3, LAC2=−2, LAC3=−1, LAC4=0, LAC5=1, LAC6=2, and LAC7=3, the respective degrees are 10, 40, 100, 200, 100, 40, and 10, and the total degree is 500, the distributions 904 of the error of the lung age reduction amount are B1=2%, B2=8%, B3=20%, B4=40%, B5=20%, B6=8%, and B7=2%. The value of the error of “0” is a center of error distribution, and, in this case, the center is LAC4=0. The information on the lung age error distribution is calculated by performing processing shown in a flowchart of FIG. 5 with the use of medical examination information of FIG. 6. Specific processing thereof will be described below.

### <Flow of Processing in System>

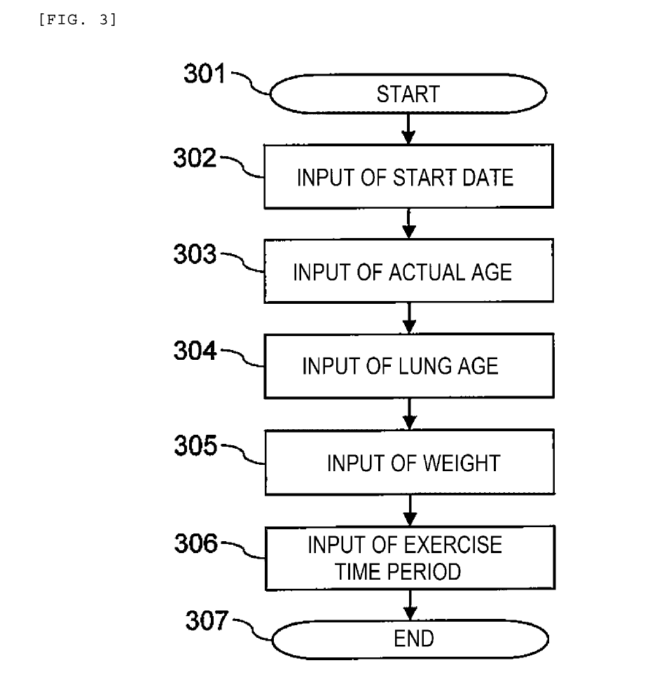

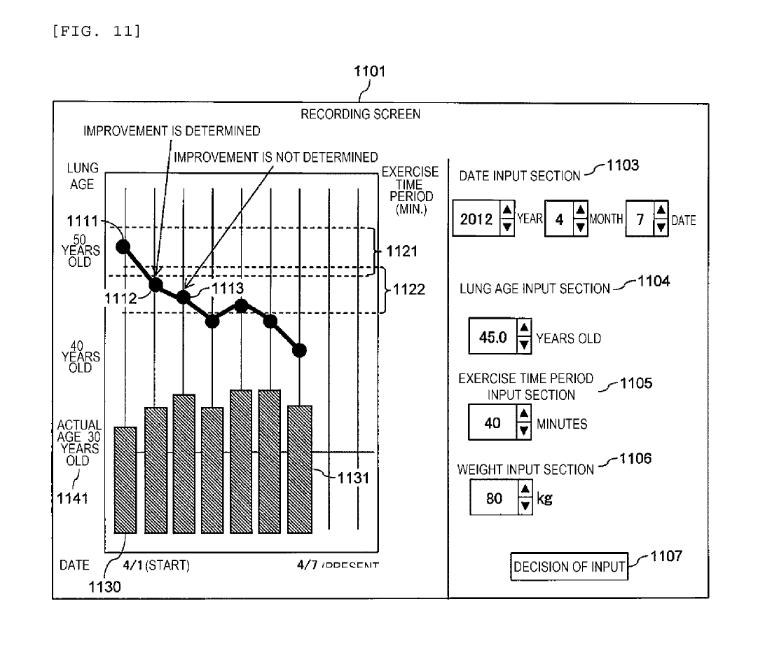

Hereinafter, a flow of processing in the exercise effect analysis system of this example will be described. FIG. 3 is a flowchart showing a flow in which information is inputted when the exercise effect analysis system is started. FIG. 10 shows an example of a user information input screen 1001 displayed when the exercise effect analysis system is started. The user information input screen 1001 includes a start date input section 1002 for inputting start date, an actual age input section 1003 for inputting an actual age, a lung age input section 1004 for inputting a lung age, a weight input section 1005 for inputting weight, an exercise time period input section 1006 for inputting an exercise time period, and an input decision button 1007 for deciding the above input.

In Step 301, when the exercise effect analysis terminal 101 of the invention is started, the exercise effect analysis terminal 101 displays the user information input screen 1001 illustrated in FIG. 10 in the output unit 104.

Next, in Step 302, the exercise effect analysis terminal 101 causes a user to input start date (year, month, and date) to the start date input section 1002 of FIG. 10 via the input unit 102.

Next, in Step 303, the exercise effect analysis terminal 101 causes the user to input an actual age to the actual age input section 1003 of FIG. 10 via the input unit 102.

Next, in Step 304, the exercise effect analysis terminal 101 causes the user to measure his/her lung age with the use of the spirometry and then causes the user to input the lung age to the lung age input section 1004 of FIG. 10 via the input unit 102. Note that the exercise effect analysis terminal 101 does not necessarily need to include the spirometry, and the user may input the lung age measured in advance.

Next, in Step 305, the exercise effect analysis terminal 101 causes the user to input weight to the weight input section 1005 of FIG. 10 via the input unit 102.

Next, in Step 306, the exercise effect analysis terminal 101 causes the user to input an exercise time period to the exercise time period input section 1006 of FIG. 10 via the input unit 102. For example, the user measures the exercise time period of walking or the like with the use of a pedometer or the like in advance and the user only needs to input a measurement value thereof. A measuring instrument such as a pedometer is not necessarily needed, and the user only needs to input an exercise time period performed at the date.

After input of the above information is completed, in Step 307, the exercise effect analysis terminal 101 causes the user to push the input decision button 1007 of FIG. 10 to thereby decide the input. The exercise effect analysis terminal 101 stores the inputted information in the recording data management unit 122 in the format shown in FIG. 7.

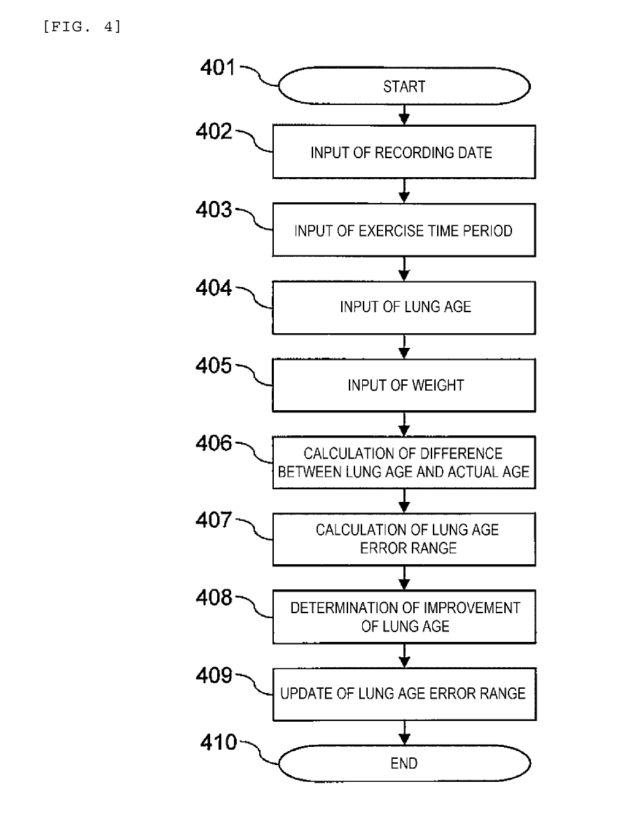

Next, a flow of processing from input of daily information of the user to determination of improvement of the lung age will be described. FIG. 4 is a flowchart showing the flow from the input of the daily information of the user to the determination of the improvement of the lung age. FIG. 11 shows an example of a recording screen 1101 for recording the daily information of the user.

The recording screen 1101 includes a date input section 1103 for inputting recording date, a lung age input section 1104 for inputting a lung age for that day, an exercise time period input section 1105 for inputting an exercise time period for that day, a weight input section 1106 for inputting weight for that day, and an input decision button 1107 for deciding the input.

The recording screen 1101 includes daily lung age graph points 1111 to 1113 and daily exercise time period graphs 1130 to 1131 inputted by the user. The lung age graph point 1111 and the exercise time period graph 1130 at the time of starting on the leftmost side are the lung age and the exercise time period of the user at the time of starting which are inputted in Steps 304 and 306 in the flowchart of FIG. 3. Further, the actual age of the user inputted in Step 303 in the flowchart of FIG. 3 is indicated as a target value 1141.

Further, error ranges 1121 and 1122 of the lung age are displayed on the recording screen 1101. The error range 1121 of the lung age shows an error range of the lung age at the lung age graph point 1111, and the error range 1122 of the lung age shows an error range of the lung age at the lung age graph point 1112. The error ranges are calculated with the use of the estimation formulae of FIG. 8 managed by the estimation formula management unit 123 and the information on the lung age error distribution of FIG. 9 managed by the lung age error distribution management unit 121. Specific processing thereof will be described below. Note that, although not shown in FIG. 11, not only the lung age graph points 1111 to 1113 of the lung age and the exercise time period graphs 1130 to 1131, but also a graph of a lung age change amount, a graph of an exercise time period change amount, and a graph of a weight change amount may be also displayed on the recording screen 1101.

Referring back to the flowchart in FIG. 4, the description will be made. In Step 401, when the exercise effect analysis terminal 101 is started, the exercise effect analysis terminal 101 displays the recording screen 1101 shown in FIG. 11 in the output unit 104.

Next, in Step 402, the exercise effect analysis terminal 101 causes the user to input recording date (year, month, and date) to the date input section 1103 of FIG. 11 via the input unit 102.

Next, in Step 403, the exercise effect analysis terminal 101 causes the user to input an exercise time period to the exercise time period input section 1105 of FIG. 11 via the input unit 102. As described above, the user measures the exercise time period of walking or the like with the use of a pedometer or the like in advance and the user may input a measurement value thereof. A measuring instrument such as a pedometer is not necessarily needed, and the user only needs to input the exercise time period performed at the date. This input result is displayed as bar graphs like the exercise time period graphs 1130 to 1131 of FIG. 11.

Next, in Step 404, the exercise effect analysis terminal 101 causes the user to measure a lung age with the use of the spirometry and causes the user to input the lung age to the lung age input section 1104 of FIG. 11 via the input unit 102. This input result is displayed like the lung age graph points 1111 to 1113 of FIG. 11. Note that the exercise effect analysis terminal 101 does not necessarily include the spirometry, and the user may input a lung age measured in advance.

Next, in Step 405, the exercise effect analysis terminal 101 causes the user to input weight to the weight input section 1106 of FIG. 11 via the input unit 102. After input processing of the above records is completed, the user is caused to push the input decision button 1107 of FIG. 11 to thereby decide the input. The exercise effect analysis terminal 101 stores the inputted information in the recording data management unit 122 in the format shown in FIG. 7.

Next, in Step 406, the subtraction calculation unit 117 subtracts the actual age from a lung age at previous recording date to calculate the difference between the lung age and the actual age (lung age−actual age). Note that, as the actual age, the actual age inputted in Step 303 of FIG. 3 only needs to be recorded and be calculated in consideration of the number of lapsed days.

Next, in Step 407, the lung age error range calculation unit 113 acquires, from the database 106, the lung age reduction amount estimation formula 801 and the lung age estimation formula 803 of FIG. 8 managed in the estimation formula management unit 123. Next, the lung age error range calculation unit 113 substitutes the difference between the lung age and the actual age (previous lung age−actual age) calculated in the subtraction calculation unit 117, the exercise time period increase amount “0”, and the weight reduction amount “0” for the lung age reduction amount estimation formula 801 and calculates an estimated lung age reduction amount caused by the difference between the lung age and the actual age which has no relation to exercise. Next, the lung age error range calculation unit 113 substitutes the estimated lung age reduction amount caused by the calculated difference between the lung age and the actual age and the previous lung age for the lung age estimation formula 803 and calculates an estimated lung age caused by the difference between the lung age and the actual age.

Next, the lung age error range calculation unit 113 acquires, from the database 106, the information on the lung age error distribution in FIG. 9 managed in the lung age error distribution management unit 121. Then, the lung age error range calculation unit 113 extracts a part including a degree having a certain ratio, from a center of the error distribution 904 (value of error: “0”) to obtain a lower limit value and an upper limit value of the error 903. For example, in a case where the certain ratio is 95%, in the example for use in the description of FIG. 9, a value of the error at the center of the distribution is LAC4=0, and therefore the lower limit value and the upper limit value in the part including the degree of 95% are LAC2=−2 and LAC6=2, respectively.

Next, the lung age error range calculation unit 113 calculates the error range of the lung age on the basis of the estimated lung age caused by the calculated difference between the lung age and the actual age and the lower limit value and the upper limit value of the error 903. The error range of the lung age is (estimated lung age−lower limit value of error 903) to (estimated lung age−upper limit value of error 903). In the above example, the lower limit value and the upper limit value in the part including the degree of 95% are LAC2=−2 and LAC6=2, respectively. Therefore, in an example where the estimated lung age is 49 years old, the error range of the lung age is from 51 years old to 47 years old. The calculated error range of the lung age is shown like the lung age error range 1121 of FIG. 11. This makes it possible to calculate the error range of reduction of the lung age caused by the difference between the lung age and the actual age which has no relation to exercise.

Next, in Step 408, the lung age improvement determination unit 114 compares a current lung age inputted in the lung age input step 404 with the error range of the lung age calculated in the lung age error range calculation unit 113. Herein, in a case where the current lung age is reduced more than (estimated lung age−upper limit value of error), the lung age improvement determination unit 114 determines that the lung age is improved. If not, the lung age improvement determination unit 114 determines that the lung age is not improved. In the example of FIG. 11, the lung age graph point 1112 is reduced more than the lung age error range 1121 with respect to the lung age graph point 1111, and therefore it is determined that the lung age is improved. This makes it possible to remove the influence of the difference between the lung age and the actual age which has no relation to exercise on the reduction of the lung age and the error thereof and accurately determine the improvement of the lung age caused by the exercise.

Next, in a case where the lung age improvement determination unit 114 determines that the lung age is improved, in Step 409, the lung age error range update unit 115 updates a display position of the lung age error range of FIG. 11. Specifically, the lung age error range is changed to a range whose center is set to a value of the lung age graph at which the improvement of the lung age is determined. As to the error range, the lung age error range calculation unit 113 performs the above processing to calculate a new range. In the example of FIG. 11, it is determined that the lung age graph point 1112 is improved with respect to the lung age graph point 1111, and therefore the lung age error range 1121 is changed to the lung age error range 1122 whose center is set to the lung age graph point 1112.

The processing of the flow from the input of the daily information of the user to the determination of the improvement of the lung age is completed (410). This processing is executed by the exercise effect analysis terminal 101 every time when the user makes a daily record.

Subsequently, creation processing of the information on the lung age error distribution of FIG. 9 will be described with the use of the medical examination information of FIG. 6 and the flowchart of FIG. 5. FIG. 5 is the flowchart showing a flow from input of the medical examination information to calculation of the lung age error distribution.

When processing of FIG. 5 is started (501), in Step 502, the exercise effect analysis terminal 101 acquires the medical examination information of FIG. 6 managed in the medical examination data management unit 120.

Next, in Step 503, the weight change calculation unit 111 calculates a weight reduction amount between two points of time at different medical examination dates for each medical examination ID 601 on the basis of the weight 604 of the acquired medical examination information of FIG. 6.

Next, in Step 504, the exercise time period change calculation unit 112 calculates an exercise time period increase amount between two points of time at different medical examination dates for each medical examination ID 601 on the basis of the exercise time period 603 of the acquired medical examination information of FIG. 6.

Next, in Step 505, the lung age change calculation unit 110 calculates a lung age reduction amount between two points of time at different medical examination dates for each medical examination ID 601 on the basis of the lung age 605 of the acquired medical examination information of FIG. 6.

Next, in Step 506, the subtraction calculation unit 117 calculates a difference between the lung age and the actual age (lung age 605−age 606) for the same medical examination date for each medical examination ID 601 on the basis of the lung age 605 and the age 606 of the acquired medical examination information of FIG. 6.

Next, in Step 507, the estimation formula creation unit 118 executes regression analysis processing by setting the lung age reduction amount calculated in the lung age change calculation unit 110 as a criterion variable and setting the exercise time period increase amount calculated in the exercise time period change calculation unit 112, the weight reduction amount calculated in the weight change calculation unit 111, and the difference between the lung age and the actual age calculated in the subtraction calculation unit 117 as explanatory variables. In this way, the lung age reduction amount estimation formula 801 of FIG. 8 is created.

Next, the estimation formula creation unit 118 executes regression analysis processing by setting the weight reduction amount calculated in the weight change calculation unit 111 as a criterion variable and setting the exercise time period increase amount calculated in the exercise time period change calculation unit 112 as an explanatory variable. In this way, the weight reduction amount estimation formula 802 of FIG. 8 is created.

Further, the estimation formula creation unit 118 creates the lung age estimation formula 803 for estimating the lung age from the previous lung age and the lung age reduction amount. The created estimation formula is registered in the format shown in FIG. 8 in the database 106 and is managed in the estimation formula management unit 123. With those estimation formulae 801, 802, and 803, the lung age reduction amount, the weight reduction amount, and the lung age can be estimated.

Next, in Step 508, the lung age error distribution calculation unit 116 acquires the lung age reduction amount estimation formula 801 managed in the estimation formula management unit 123. Next, members of the exercise time period increase amount and the weight reduction amount of the lung age reduction amount estimation formula 801 are transposed to the left side. Thus, the following formula is created.

Lung age reduction amount−A×exercise time period increase amount−B×weight reduction amount=C×difference between lung age and actual age+E

Next, the lung age error distribution calculation unit 116 substitutes, for the created formula, the lung age reduction amount calculated in the lung age change calculation unit 110, the weight reduction amount calculated in the weight change calculation unit 111, the exercise time period increase amount calculated in the exercise time period change calculation unit 112, and the difference between the lung age and the actual age calculated in the subtraction calculation unit 117. In this way, in the created formula, values on the right side and the left side are calculated for each medical examination ID 601. Then, distribution of a subtraction between the value on the right side and the value on the left side is obtained, and then error distribution of the lung age is calculated. Herein, the value on the right side indicates a lung age reduction effect caused by the difference between the lung age and the actual age which has no relation to exercise and the value on the left side indicates a value obtained by removing, from an actual lung age reduction amount, a reduction effect caused by the exercise time period and a reduction effect via the weight. By calculating the distribution of the subtraction between the value on the right side and the value on the left side of the created formula as described above, it is possible to create error distribution of the lung age reduction amount caused by the difference between the lung age and the actual age which has no relation to exercise.

The calculation processing of the lung age error distribution is completed (509). The calculated lung age error distribution is registered in the format shown in FIG. 9 in the database 106 and is managed in the lung age error distribution management unit 121.

As described above, the exercise effect analysis system of this example can accurately determine the improvement of the lung age caused by exercise on the basis of the lung age and the lung age error distribution. Therefore, only by inputting the exercise information and the lung age information that can be easily measured by a user, it is possible to accurately determine a degree of improvement of the lung age caused by exercise and analyze and display the exercise effect of the user.

The invention is not limited to the above example and encompasses various modification examples. For example, the above example has been described in detail to easily understand the invention and the invention is not necessarily limited to a system having all the configurations described above. Further, another configuration can be added to/removed from/replaced with a part of the configuration in each example.

For example, in order to input exercise information and lung age information that can be easily measured to know improvement of a lung age caused by exercise, the exercise effect analysis system only needs to include at least the input unit 102, the exercise time period change calculation unit 112, the lung age change calculation unit 110, and the output unit 104. For example, exercise information and lung age information of a user are inputted with the use of the input unit 102. Then, the exercise time period change calculation unit 112 calculates an exercise time period change amount that is a difference between first exercise information and second exercise information. The lung age change calculation unit 110 calculates a lung age change amount that is a difference between first lung age information and second lung age information. The output unit 104 displays the lung age change amount and at least one of the exercise information of the user and the exercise time period change amount. The above example is a more preferable embodiment of the invention, and the invention can be formed by removing a part of the configuration described above.

The above example has described an example of causing a user to measure a lung age with the use of a spirometry and to daily record the lung age. However, the lung age may be estimated from an exercise time period and weight which are daily recorded. For example, the exercise effect analysis unit 105 may include a lung age estimation unit for estimating a lung age from an exercise time period and weight information. The lung age estimation unit estimates the lung age with the use of the lung age reduction amount estimation formula 801 and the lung age estimation formula 803 of FIG. 8 managed by the estimation formula management unit 123. Specifically, the exercise time period change calculation unit 112 calculates an exercise time period increase amount on the basis of an exercise time period of a user for start date and a current exercise time period. Then, the weight change calculation unit 111 calculates a weight reduction amount on the basis of previous weight and current weight. Next, the subtraction calculation unit 117 calculates a difference between a lung age and an actual age on the basis of a previous estimated lung age and the actual age. Then, the calculated exercise time period increase amount, the weight reduction amount, and the difference between the lung age and the actual age are substituted for the lung age reduction amount estimation formula 801 to calculate an estimated lung age reduction amount. Further, the calculated estimated lung age reduction amount and the previous estimated lung age are substituted for the lung age estimation formula 803 to calculate and display an estimated lung age. By estimating the lung age from the exercise time period and the weight as described above, a user can save time and labor for measuring and recording a daily lung age.

Further, recording of the weight may be also omitted and the lung age may be estimated only from the exercise time period. The lung age estimation unit estimates the lung age with the use of not only the lung age reduction amount estimation formula 801 and the lung age estimation formula 803 of FIG. 8 but also the weight reduction amount estimation formula 802. Specifically, the exercise time period increase amount calculated in the exercise time period change calculation unit 112 is substituted for the weight reduction amount estimation formula 802 to calculate an estimated weight reduction amount. Then, the estimated weight reduction amount is used instead of an actual weight reduction amount, the estimated lung age reduction amount and the estimated lung age are calculated and displayed as described above. By estimating the lung age only on the basis of the exercise time period as described above, a user can save time and labor for measuring and recording not only the daily lung age but also weight.

Further, in the above example, a lung age measured and recorded by a user and a lung age error range are compared with each other and whether or not the lung age is improved is simply determined with the use of binary values (binary values of “improved” and “not improved”). However, this determination may be performed in another way. For example, the lung age improvement determination unit 114 may calculate a degree of improvement (display showing what percentage the lung age is improved) by comparing a previous lung age with a current lung age and may display a comment corresponding to the degree of improvement. This can be achieved in the following way: a lung age reduction amount caused by exercise is obtained by subtracting an estimated lung age caused by a difference between a lung age calculated in the lung age error range calculation unit 113 and an actual age from a lung age measured by a user or an estimated lung age; and the lung age reduction amount is compared with the lung age error distribution information of FIG. 9.

Specifically, a cumulative ratio of the error distribution 904 of the lung age reduction amount from a minimum value of an error of the lung age reduction amount to the calculated lung age reduction amount caused by exercise is obtained and this value is displayed as the degree of improvement. For example, in a case where the lung age reduction amount caused by exercise is 1, in the example of the description of FIG. 9 described above, the cumulative ratio of the error distribution from the minimum value LAC1=−3 to LAC5=1 is calculated, which is B1+B2+B3+B4+B5=90%, and is displayed as the degree of improvement. Further, a table in which the degree of improvement and a corresponding comment are stored is prepared, and a comment such as “The lung age will be improved soon.” is displayed in accordance with the calculated degree of improvement. By calculating and displaying the degree of improvement and the comment corresponding to the degree of improvement as described above, it is possible to quantitatively display an effect of exercise of a user in more detail.

Further, in addition to the above example, the lung age improvement determination unit 114 may calculate an exercise time period increase amount needed to improve a lung age of a user to an actual age or a target value that has been set in advance by the user and display the exercise time period increase amount in the output unit 104. This can be achieved by using the lung age reduction amount estimation formula 801 and the weight reduction amount estimation formula 802 of FIG. 8. Specifically, the exercise time period increase amount can be calculated by the following formula that is created by changing the lung age reduction amount estimation formula 801 and the weight reduction amount estimation formula 802.

Exercise time period increase amount=(target lung age reduction amount−C×difference between lung age and actual age−E−B×F)/(A+B×D)

This makes it possible to display how much time the user needs to exercises in order to improve the lung age to the target value, and therefore a daily target of exercise can be easily planned. The above example has described an example of measuring and recording the exercise time period. However, an amount of exercise (cal) or the number of steps may be measured and recorded.

The estimation formulae are not limited to those of FIG. 8. Another estimation formula may be created by using the relationship of FIG. 2. For example, the estimation formula may be created in consideration of the influence 211 of the difference between the lung age and the actual age on the lung age reduction amount and the influence 212 of the exercise time period increase amount on the lung age reduction amount shown in FIG. 2. For example, the estimation formula creation unit 118 may execute regression analysis processing by setting the lung age change amount as a criterion variable and setting the exercise amount change amount and the difference between the lung age and the actual age as explanatory variables, thereby creating an estimation formula. In this case, the lung age error distribution calculation unit 116 may substitute, for the created estimation formula, the difference between the lung age and the actual age, the lung age change amount, and the exercise amount change amount, thereby creating lung age error distribution information.

As described above, the configuration of the example can be realized with the use of hardware by, for example, designing a part or all thereof with an integrated circuit. Further, the invention may be realized by a program code of software that realizes a function of the example. In this case, a storage medium on which the program code is recorded is provided to an information processing apparatus and the information processing apparatus (or CPU) reads the program code stored in the storage medium. In this case, the program code itself read from the storage medium realizes the function of the above example, and the program code itself and the storage medium in which the program code is stored form the invention.

Furthermore, by distributing the program code of the software that realizes the function of the example via a network, the program code is stored in a storage device of the information processing apparatus or a storage medium such as CD-RW or CD-R, and, at the time of using the program code, a CPU of the information processing apparatus may read the program code stored in the storage device or the storage medium and execute the program code.

### REFERENCE SIGNS LIST

101 exercise effect analysis terminal
102 input unit
104 output unit
105 exercise effect analysis unit
106 database
110 lung age change calculation unit
111 weight change calculation unit
112 exercise time period change calculation unit
113 lung age error range calculation unit
114 lung age improvement determination unit
115 lung age error range update unit
116 lung age error distribution calculation unit
117 subtraction calculation unit
118 estimation formula creation unit
120 medical examination data management unit
121 lung age error distribution management unit
122 recording data management unit
123 estimation formula management unit
201 difference between lung age and actual age
202 exercise time period increase amount
203 weight reduction amount
204 lung age reduction amount
211 influence of difference between lung age and actual age on lung age reduction amount
212 influence of exercise time period increase amount on lung age reduction amount
213 influence of exercise time period increase amount on weight reduction amount
214 influence of weight reduction amount on lung age reduction amount
302 start date input step
303 actual age input step
304 lung age input step
305 weight input step
306 exercise time period input step
406 calculation step of difference between lung age and actual age
407 lung age error range calculation step
408 lung age improvement determination step
409 lung age error range update step
502 medical examination data input step
503 weight change calculation step
504 exercise time period change calculation step
505 lung age change calculation step
506 calculation step of difference between lung age and actual age
507 estimation formula creation step
508 lung age error distribution calculation step
601 medical examination ID
602 medical examination date
603 exercise time period
604 weight
605 lung age
606 age
701 user ID
702 recording date
703 exercise time period
704 weight
705 lung age
801 lung age reduction amount estimation formula
802 weight reduction amount estimation formula
803 lung age estimation formula
903 error of lung age reduction amount
904 error distribution of lung age reduction amount
1001 user information input screen
1002 start date input section
1003 actual age input section
1004 lung age input section
1005 weight input section
1006 exercise time period input section
1007 input decision button
1101 recording screen
1103 date input section
1104 lung age input section
1105 exercise time period input section
1106 weight input section
1107 input decision button
1111 to 1113 lung age recording graph
1121 to 1122 lung age error range
1131 exercise time period
1141 actual age (target value)

1. An exercise effect analysis system for analyzing and displaying an exercise effect on the basis of lung function information of a user, comprising:
an input unit for inputting at least exercise information and lung age information of the user;
an exercise amount change calculation unit for calculating an exercise amount change amount that is a difference between first exercise information and second exercise information inputted in the input unit;
a lung age change calculation unit for calculating a lung age change amount that is a difference between first lung age information and second lung age information; and
an output unit for displaying the lung age change amount and at least one of the exercise information of the user and the exercise amount change amount.

2. The exercise effect analysis system according to claim 1, further comprising:
a subtraction calculation unit for calculating a difference between a lung age and an actual age, the difference being a difference between age information and the lung age information of the user;
a storage device in which user recording information in which at least the exercise information and the lung age information of the user are recorded, an estimation formula showing a relationship among the exercise information, the age, the lung age, and an estimated lung age change amount, and lung age error distribution information indicating distribution of errors of lung age change amounts of a plurality of users are stored;
a lung age error range calculation unit for substituting the difference between the lung age and the actual age for the estimation formula to calculate an estimated lung age and calculating a lung age error range on the basis of the estimated lung age and the lung age error distribution information; and
a lung age improvement determination unit for comparing the lung age change amount with the lung age error range to determine improvement of the lung age of the user,
wherein the output unit displays the lung age change amount, the lung age error range, and at least one of the exercise information of the user recording information and the exercise amount change amount.

3. The exercise effect analysis system according to claim 2, further comprising
a lung age estimation unit for estimating the second lung age information from the exercise amount change amount, wherein:
the subtraction calculation unit calculates the difference between the lung age and the actual age, the difference being a difference between the lung age information recorded in the user recording information and the actual age;
the lung age estimation unit substitutes the difference between the lung age and the actual age and the exercise amount change amount for the estimation formula to estimate the second lung age information; and
the lung age change calculation unit calculates, as the lung age change amount, a difference between the first lung age information and the estimated second lung age information.

4. The exercise effect analysis system according to claim 2, further comprising
an estimation formula creation unit for creating the estimation formula, wherein
the estimation formula creation unit executes regression analysis processing by setting the lung age change amount as a criterion variable and setting the exercise amount change amount and the difference between the lung age and the actual age as explanatory variables to create the estimation formula.

5. The exercise effect analysis system according to claim 2, further comprising
a lung age error distribution calculation unit for substituting the difference between the lung age and the actual age, the lung age change amount, and the exercise amount change amount for the estimation formula to create the lung age error distribution information.

6. The exercise effect analysis system according to claim 2, further comprising
a lung age error range update unit for updating the lung age error range in a case where the lung age improvement determination unit determines that the lung age of the user is improved.

7. The exercise effect analysis system according to claim 2, wherein:
the lung age improvement determination unit calculates, as a degree of improvement, a cumulative ratio within a predetermined range in the lung age error distribution information; and
the output unit further displays the degree of improvement.

8. The exercise effect analysis system according to claim 2, wherein:
the lung age improvement determination unit substitutes the difference between the lung age and the actual age and a target lung age change amount set by the user for the estimation formula to calculate a necessary exercise amount change amount needed to improve the target lung age change amount; and
the output unit further displays the necessary exercise amount change amount.

9. The exercise effect analysis system according to claim 1, further comprising:
a weight change calculation unit for calculating a weight change amount that is a difference between first weight information and second weight information;
a subtraction calculation unit for calculating a difference between a lung age and an actual age, the difference being a difference between age information and the lung age information of the user;
a storage device in which user recording information in which at least the exercise information, the weight information, and the lung age information of the user are recorded, an estimation formula showing a relationship among the exercise information, the weight information, the age, the lung age, and an estimated lung age change amount, and lung age error distribution information indicating distribution of errors of lung age change amounts of a plurality of users are stored;
a lung age error range calculation unit for substituting the difference between the lung age and the actual age for the estimation formula to calculate an estimated lung age and calculating a lung age error range on the basis of the estimated lung age and the lung age error distribution information; and
a lung age improvement determination unit for comparing the lung age change amount with the lung age error range to determine improvement of the lung age of the user,
wherein the output unit displays the lung age change amount, the lung age error range, and at least one of the exercise information of the user recording information and the exercise amount change amount.

10. The exercise effect analysis system according to claim 9, further comprising
a lung age estimation unit for estimating the second lung age information from the exercise amount change amount and the weight change amount, wherein:
the subtraction calculation unit calculates the difference between the lung age and the actual age, the difference being a difference between the lung age information recorded in the user recording information and the actual age;
the lung age estimation unit substitutes the difference between the lung age and the actual age, the exercise amount change amount, and the weight change amount for the estimation formula to estimate the second lung age information; and
the lung age change calculation unit calculates, as the lung age change amount, a difference between the first lung age information and the estimated second lung age information.

11. The exercise effect analysis system according to claim 9, further comprising
an estimation formula creation unit for creating the estimation formula, wherein
the estimation formula creation unit executes regression analysis processing by setting the lung age change amount as a criterion variable and setting the exercise amount change amount, the weight change amount, and the difference between the lung age and the actual age as explanatory variables to create the estimation formula.

12. The exercise effect analysis system according to claim 9, further comprising
a lung age error distribution calculation unit for substituting the difference between the lung age and the actual age, the lung age change amount, the exercise amount change amount, and the weight change amount for the estimation formula to create the lung age error distribution information.

13. The exercise effect analysis system according to claim 9, further comprising
a lung age error range update unit for updating the lung age error range in a case where the lung age improvement determination unit determines that the lung age of the user is improved.

14. The exercise effect analysis system according to claim 9, wherein:
the lung age improvement determination unit calculates, as a degree of improvement, a cumulative ratio within a predetermined range in the lung age error distribution information; and
the output unit further displays the degree of improvement.

15. The exercise effect analysis system according to claim 9, wherein:
the lung age improvement determination unit substitutes the difference between the lung age and the actual age and a target lung age change amount set by the user for the estimation formula to calculate a necessary exercise amount change amount needed to improve the target lung age change amount; and
the output unit further displays the necessary exercise amount change amount.
